(19) United States
(12) Patent Application Publication
(10) Pub. No.: US 2024/0058621 A1
(43) Pub. Date: Feb. 22, 2024

(54) PHOTOTHERAPY CAP AND LINING THEREOF

(71) Applicant: Chung-Yang CHEN, New Taipei City (TW)

(72) Inventor: Chung-Yang CHEN, New Taipei City (TW)

(21) Appl. No.: 17/892,562

(22) Filed: Aug. 22, 2022

(51) Int. Cl. A61N 5/06

(52) U.S. Cl. CPC A61N 5/062; A61N 5/0616; A61N 2005/0645

(57) ABSTRACT

A phototherapy lining, assembled to a cap body, includes a composite substrate, a plurality of light-emitting units, and a fixing assembly. The composite substrate includes a first flexible substrate, a second flexible substrate and a flexible printed circuit layer. The first flexible substrate is adhered to the cap body. The second flexible substrate is transparent and disposed close to a user's head. The second flexible substrate includes a second central portion, a plurality of second extension portions and a plurality of hook portions. The hook portions are utilized to cover part of the first flexible substrate. The flexible printed circuit layer is disposed between the first flexible substrate and the second flexible substrate. The light-emitting units are utilized to irradiate a plurality of beams to the user's head. The fixing assembly is utilized to fix the composite substrate.

### BACKGROUND OF THE INVENTION

### (1) Field of the Invention

The invention relates to a cap and a lining of the cap, and more particularly to a wearable head phototherapy cap and a lining thereof.

### (2) Description of the Prior Art

In order to solve the problem of baldness, a variety of different treatments have been developed. For example, a topical lotion is introduced to be applied onto a bald part of a head, and this lotion can be a peripheral vasodilator for improving blood circulation and nutrient supply of the corresponding scalp on the head, so that the hair follicles can re-grow. In another example, an oral medicine is introduced to inhibit the male hormonal so as to prevent hair follicles from shrinking. However, the two above-mentioned methods may be provided with specific side effects on human body to some extent, from which health of the human body may be affected. In particular, residues of these medicines may affect health of subsequent pregnancy.

In addition, a treatment method by performing a hair transplant operation is introduced. Although this method may restore user's appearance, yet the original hair will continue to fall out, and the cost of hair transplant operation is expensive. Therefore, an improvement thereto is definitely necessary. Further, a laser treatment is also developed, but the cost of associated equipment is high. Thus, this laser treatment can be only performed in medical institutions. Also, the related laser equipment is heavy, and thus can't be directly and comfortably applied to user's head. Namely, use of such a laser treatment is limited and inconvenient.

Furthermore, due to high work pressure, poor eating habits and/or abnormal long-term work and rest, problems related to scalp health, hair growth and the like abnormality would become issues. If any of headaches, insomnia, mental depression and a problem the like is met, medication or injection is definitely insufficient, because some significant side effects would affect daily life and lead to unexpected health burden in the long run. Thus, a great need for a non-drug or injection phototherapy cap is emergent.

### SUMMARY OF THE INVENTION

In view that some specific side effects caused by the medication treatment upon baldness in the prior art may affect human health, even possible pregnancies, thus improvement thereto is required. In addition, the traditional hair transplant surgery is expensive but ways to solve the original hair loss problem, also the heavy weight of related laser equipment causes a portability problem, and thus the use thereof is definitely limited and inconvenient. Further, ill eating, work and rest habits would lead to problems in scalp health, hair growth, headache, insomnia, and mental distress. Accordingly, a purpose of this present invention is to provide a wearable phototherapy cap and a lining thereof to resolve at least one of the aforesaid shortcomings in the art.

In this invention, a phototherapy lining, assembled to an inner surface of a cap body, includes a composite substrate, a plurality of light-emitting units and a fixing assembly. The composite substrate has a composite central portion, and includes a first flexible substrate, a second flexible substrate and a flexible printed circuit layer. The first flexible substrate is adhered to the inner surface. The second flexible substrate, disposed away from the inner surface with respect to the first flexible substrate, is flexible, transparent and configured to be worn on a user's head. The second flexible substrate includes a second central portion, a plurality of second extension portions and a plurality of hook portions. The second extension portion is extended from the second central portion. The hook portion is reflexed respectively from the corresponding second extension portion to wrap part of the first flexible substrate. The flexible printed circuit layer is disposed between the first flexible substrate and the second flexible substrate. Each of the light-emitting units is disposed between the flexible printed circuit layer and the second flexible substrate, and configured to generate a light beam to irradiate the user's head. The fixing assembly, disposed at the composite central portion, is configured for fixing the first flexible substrate, the flexible printed circuit layer and the second flexible substrate.

In one embodiment of this present invention, the first flexible substrate further includes a first central portion and a plurality of first extension portions extending from the first central portion.

In one embodiment of this present invention, the flexible printed circuit layer further includes a third central portion and a plurality of third extension portions extending from the third central portion.

In one embodiment of this present invention, the first extension portions are separated to each other, the second extension portions are separated to each other, and the third extension portions are separated to each other.

In one embodiment of this present invention, the second flexible substrate further includes a plurality of lateral extension portions, each of the lateral extension portions is extended toward the first flexible substrate from the corresponding second extension portion, and thereby the first flexible substrate and the second flexible substrate are integrated to wrap the flexible printed circuit layer.

In one embodiment of this present invention, one of the third extension portions is longer than any of the rest of the third extension portions, and the second extension portion corresponding to the one of the third extension portions is longer than any of the rest of the second extension portions; wherein, while being applied to the user's head, one of the third extension portions is disposed in correspondence to a lower rear area of the user's head.

In one embodiment of this present invention, the light-emitting units are disposed at the flexible printed circuit layer in an equal-spaced manner.

In one embodiment of this present invention, the light beam has a wavelength ranging between 600 nm and 1000 nm.

In another aspect of this invention, a wearable phototherapy cap, by a user, includes a cap body and a phototherapy lining. The cap body has an inner surface. The phototherapy lining, assembled to the inner surface, includes a composite substrate, a plurality of light-emitting units and a fixing assembly. The composite substrate has a composite central portion, and includes a first flexible substrate, a second flexible substrate and a flexible printed circuit layer. The first flexible substrate is adhered to the inner surface. The second flexible substrate, disposed away from the inner surface with respect to the first flexible substrate, is flexible, transparent and configured to be worn on a user's head. The second flexible substrate includes a second central portion, a plurality of second extension portions and a plurality of hook portions. The second extension portion is extended from the second central portion. The hook portion is reflexed respectively from the corresponding second extension portion to wrap part of the first flexible substrate. The flexible printed circuit layer is disposed between the first flexible substrate and the second flexible substrate. Each of the light-emitting units is disposed between the flexible printed circuit layer and the second flexible substrate, and configured to generate a light beam to irradiate the user's head. The fixing assembly, disposed at the composite central portion, is configured for fixing the first flexible substrate, the flexible printed circuit layer and the second flexible substrate.

In one embodiment of this present invention, the cap body further includes at least one wrap portion extending from a body portion of the cap body by opposing the inner surface, and the at least one wrap portion is configured to wrap auxiliarily at least part of the phototherapy lining, such that the at least part of the phototherapy lining is prevented from directly contacting a lower rear area of the user's head and used to cover auxiliarily the lower rear area.

As stated, in the wearable head phototherapy cap and the lining thereof provided by this invention, the first flexible substrate, the second flexible substrate, the flexible printed circuit layer, the light-emitting units and the fixing assembly are integrated for the user to stimulate and promote the hair growth. This invention further utilizes the hook portion of the second flexible substrate to reflex and further extend to wrap part of the first flexible substrate, such that the flexible printed circuit layer and the first flexible substrate can be free from contacting the user to cause discomfort. In addition, since the first flexible substrate, the second flexible substrate and the flexible printed circuit layer are flexible, and the first extension portion, the second extension portion and the third extension portion are separated to each other, thus the cap of the invention can fit various sizes of different users. Further, the wrap portion of the cap body is further used for wrapping auxiliarily part of the phototherapy lining, contacting the phototherapy lining, and thereby fixing auxiliarily the phototherapy lining and the cap body. The wrap portion and the hook portion are integrated to prevent user's sweat from entering the flexible printed circuit layer, and thus possible damages to the flexible printed circuit layer and the light-emitting units can be avoided. In addition, one of the third extension portions and the corresponding second extension portion are longer than the rest thereof, such that user's head, especially the lower rear portion, can be further irradiated.

All these objects are achieved by the phototherapy cap and the lining thereof described below.

### BRIEF DESCRIPTION OF THE DRAWINGS

The present invention will now be specified with reference to its preferred embodiment illustrated in the drawings, in which:

FIG. 1 is a schematic perspective view of an embodiment of the wearable phototherapy cap in accordance with this invention;

FIG. 2 shows schematically another view of FIG. 1;

FIG. 3 is a schematic exploded view of FIG. 2;

FIG. 4 shows schematically another exploded view of FIG. 2;

FIG. 5 is a schematic perspective view of the second flexible substrate of the embodiment of FIG. 1;

FIG. 6 is a schematic perspective view of the flexible printed circuit layer and the light-emitting units of the embodiment of FIG. 1;

FIG. 7 is a schematic perspective view of the first flexible substrate of the embodiment of FIG. 1;

FIG. 8 is a schematic perspective view of the cap body of the embodiment of FIG. 1;

FIG. 9 is a schematic cross-sectional view of FIG. 1 along line B-B; and

FIG. 10 demonstrates schematically a use state of the wearable phototherapy cap of FIG. 1.

### DESCRIPTION OF THE PREFERRED EMBODIMENT

The invention disclosed herein is directed to a phototherapy cap and a lining thereof. In the following description, numerous details are set forth in order to provide a thorough understanding of the present invention. It will be appreciated by one skilled in the art that variations of these specific details are possible while still achieving the results of the present invention. In other instance, well-known components are not described in detail in order not to unnecessarily obscure the present invention.

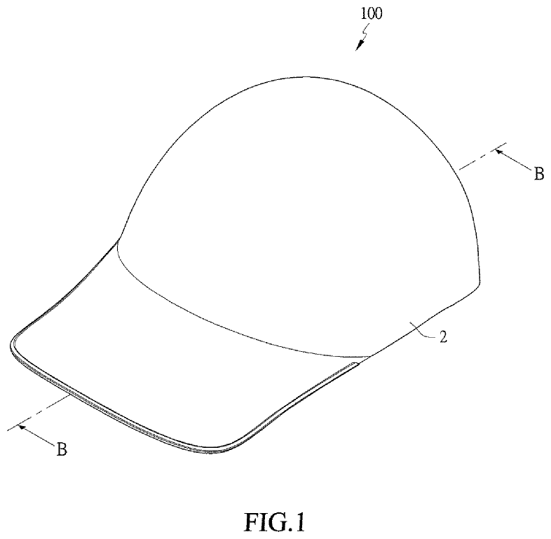

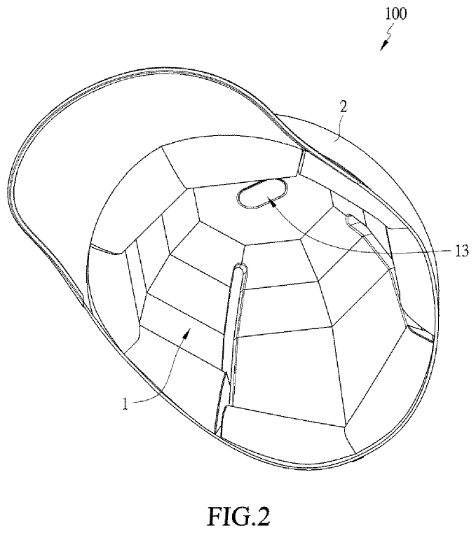

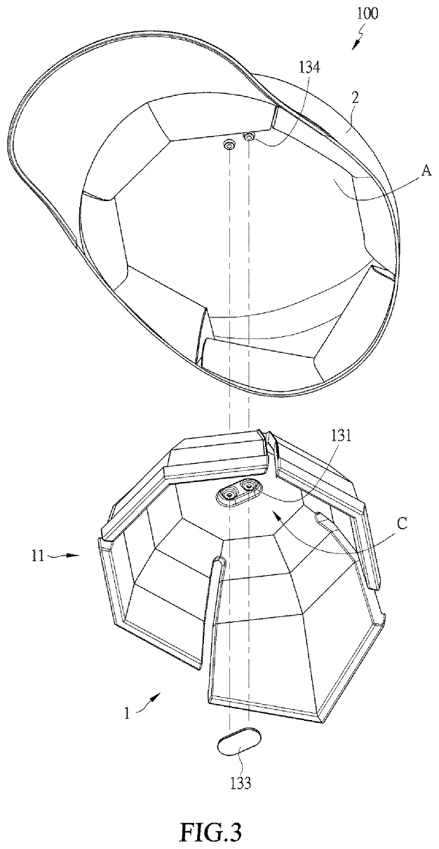

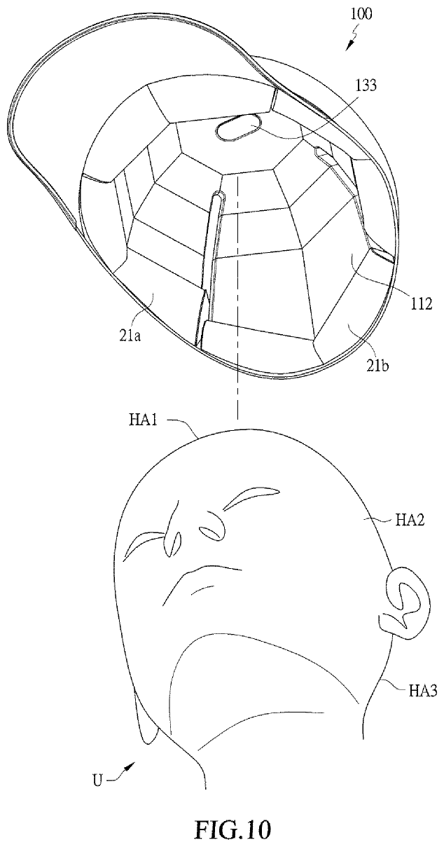

Refer to FIG. 1 through FIG. 3; where FIG. 1 is a schematic perspective view of an embodiment of the wearable phototherapy cap in accordance with this invention, FIG. 2 shows schematically another view of FIG. 1, and FIG. 3 is a schematic exploded view of FIG. 2. As shown, the wearable phototherapy cap 100, configured to be worn by a user U (labeled in FIG. 10), includes a phototherapy lining 1 and a cap body 2.

The cap body 2 has an inner surface A. In this embodiment, the cap body 2 has an appearance similar to a baseball cap, and thus the cap body 2 is provided with a sun-shielding function like the baseball cap does.

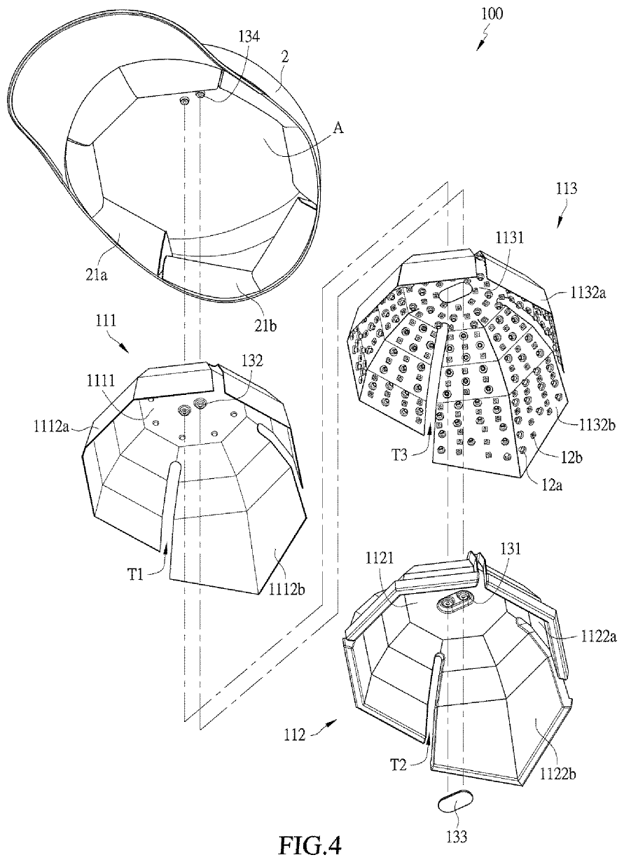

The phototherapy lining 1, assembled onto the inner surface A of the cap body 2, includes a composite substrate 11, a plurality of light-emitting units 12a, 12b (labeled in FIG. 4) and a fixing assembly 13.

The composite substrate 11 has a composite central portion C. The fixing assembly 13 is disposed at the composite central portion C to fix the composite substrate 11. In this embodiment, the fixing assembly 13 is further utilized to fix the composite substrate 11 to the inner surface A of the cap body 2.

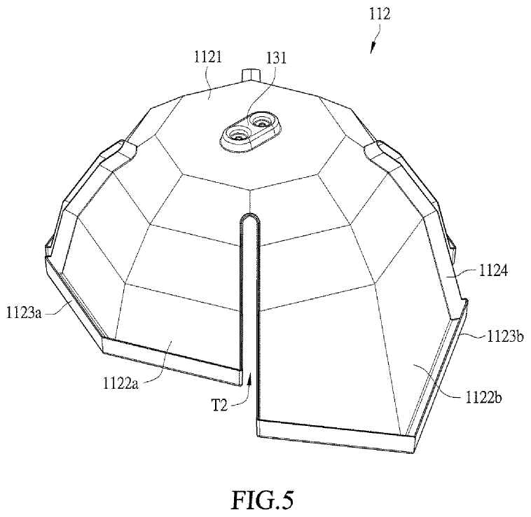

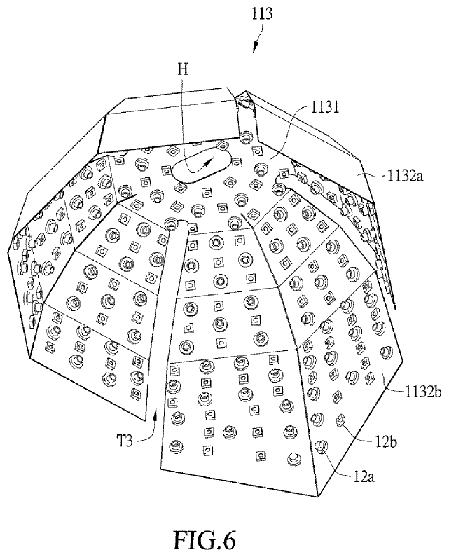

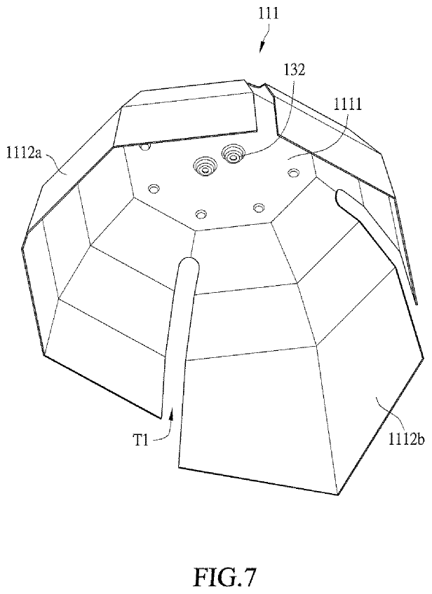

Refer to FIG. 2 through FIG. 7 together; where FIG. 4 shows schematically another exploded view of FIG. 2, FIG. 5 is a schematic perspective view of the second flexible substrate of the embodiment of FIG. 1, FIG. 6 is a schematic perspective view of the flexible printed circuit layer and the light-emitting units of the embodiment of FIG. 1, and FIG. 7 is a schematic perspective view of the first flexible substrate of the embodiment of FIG. 1. As shown, the composite substrate 11 further includes a first flexible substrate 111, a second flexible substrate 112 and a flexible printed circuit layer 113.

While in assembling, the first flexible substrate 111 is adhered to the inner surface A of the cap body 2. The first flexible substrate 111 is flexible, and thus able to match a head profile of the user U (labeled in FIG. 10). That is, since the first flexible substrate 111 is flexible, thus, while the user U wears the phototherapy cap 100, the first flexible substrate 111 can fit the head of the user U. Namely, the first flexible substrate 111 can be applied to various head sizes of different users U.

The second flexible substrate 112, disposed away from the inner surface A with respect to the first flexible substrate 111, is applied directly to fit the user U. The second flexible substrate 112 can be transparent and flexible, and thus conformable to different users U. Similarly, with considerable flexibility, the second flexible substrate 112 can deform to fix different sizes of the heads, and thus the first flexible substrate 111 can be also applied by different users U. Namely, the phototherapy lining 1 of this invention is provided with malleability. In this embodiment, the second flexible substrate 112 further includes a second central portion 1121, a plurality of second extension portions 1122a, 1122b (two labeled in the figure) and a plurality of hook portions 1123a, 1123b (two labeled in the figure).

The second central portion 1121, disposed respective to the composite central portion C, protrudes outward the second extension portions 1122a, 1122b therefrom. In this embodiment, a length of the second extension portion 1122b is greater than that of any other second extension portion 1122a. Basically, the second extension portion 1122b is disposed away from a brim of the cap body 2, but not limited thereto. In some other embodiments, the length of the second extension portion 1122b can be equal to that of any of the other second extension portions 1122a.

Each of the hook portions 1123a, 1123b is reflexed from the respective second extension portion 1122a or 1122b so as to wrap a corresponding portion of the first flexible substrate 111. It shall be explained that the reflex extension herein indicates the extension directions of the hook portions 1123a, 1123b is different to those of the corresponding second extension portions 1122a, 1122b. Preferably, angles between the hook portions 1123a, 1123b and the corresponding second extension portions 1122a, 1122b are less than 90°.

The flexible printed circuit layer 113, disposed between the first flexible substrate 111 and the second flexible substrate 112, is one of ordinary skills in the art, and thus details thereabout would be omitted herein.

The light-emitting units 12a, 12b are disposed on the flexible printed circuit layer 113, are located on a surface of the flexible printed circuit layer 113 facing the second flexible substrate 112. Each of the light-emitting units 12a, 12b would generate a light beam, and thus the light-emitting units will generate a plurality of light beams. It shall be explained that the light-emitting units 12a, 12b can be identical or different. In the figure, two structures (labeled as 12a and 12b, respectively) are provided to the light-emitting units, but not limited thereto. In this invention, each of the light-emitting units 12a, 12b can be an LED, a solid-state laser, a semiconductor laser or any the like that can generate a light beam. In this embodiment, the light beam has a wavelength ranging between 600 nm and 1000 nm. Preferably, these light-emitting units 12a, 12b are equally spaced on the flexible printed circuit layer 113.

In this embodiment, the first flexible substrate 111 further includes a first central portion 1111 and a plurality of first extension portions 1112a, 1112b individually extended from the first central portion 1111. Structurally, the first extension portion 1112b is longer than the first extension portion 1112a. Particularly, the first extension portion 1112a is disposed in correspondence to the second extension portion 1122a, and the first extension portion 1112b is disposed in correspondence to the second extension portion 1122b.

The flexible printed circuit layer 113 further includes a third central portion 1131 and a plurality of third extension portions 1132a, 1132b extending individually from the third central portion 1131. Particularly, the third extension portion 1132b is longer than the third extension portion 1132a, and the third extension portions 1132a, 1132b are disposed in correspondence to the first extension portion 1112a and the second extension portion 1122a, respectively. In addition, the first central portion 1111, the second central portion 1121 and the third central portion 1131 are integrated to form the composite central portion C of the composite substrate 11.

In this embodiment, the second extension portions 1122a, 1122b are separated to each other, the first extension portions 1112a, 1112b are separated to each other, and the third extension portions 1132a, 1132b are also separated to each other as well. The neighboring first extension portions 1112a, 1112b are spaced by a first gap T1, the neighboring second extension portion 1122a, 1122b are spaced by a second gap T2, and the neighboring third extension portions 1132a, 1132b are spaced by a third gap T3. In addition, with the first gaps T1, the second gaps T2 and the third gaps T3, while the first flexible substrate 111, the second flexible substrate 112 and the flexible printed circuit layer 113 are fitted to the head of user U (labeled in FIG. 10), possible structural interference at the first flexible substrate 111, the second flexible substrate 112 and the flexible printed circuit layer 113 can be completely voided.

The second flexible substrate 112 further includes a plurality of lateral extension portions 1124 extending from the corresponding second extension portions 1122a, 1122b toward the first flexible substrate 111, such that the second flexible substrate 112 and the first flexible substrate 111 together can hold the flexible printed circuit layer 113. As shown, each of the lateral extension portions 1124 would be extended from the corresponding second extension portion 1122a or 1122b to shield orderly the second gap T2, the third gap T3 and the first gap T1, and to protrude over the first flexible substrate 111. Thereupon, the lateral extension portions 1124 can prevent possible sweat of the user U from reaching the flexible printed circuit layer 113 via the corresponding second gaps T2 and the third gaps T3.

The fixing assembly 13, disposed at the composite central portion C, is configured for fixing the first flexible substrate 111, the second flexible substrate 112 and the flexible printed circuit layer 113. In this embodiment, the fixing assembly 13 further includes a first fixing member 131, a second fixing member 132 (formed as a pair in FIG. 4), a cover plate 133 and a cap-body fixing member 134. The first fixing member 131 is fixed at the second flexible substrate 112, and the second fixing member 132 is fixed at the first flexible substrate 111. The flexible printed circuit layer 113 is furnished with a penetration hole H in correspondence to the first fixing member 131 and the second fixing member 132. The second fixing member 132 at the first flexible substrate 111 would penetrate through the penetration hole H of the flexible printed circuit layer 113 and be then fixed to the first fixing member 131 of the second flexible substrate 112. The cover plate 133 is configured to shield the first fixing member 131, so as to prevent the first fixing member 131 from directly touching the user U (labeled in FIG. 10). In addition, the hook portions 1123a, 1123b would wrap part of the first flexible substrate 111, and also part of the corresponding flexible printed circuit layer 113, such that the first flexible substrate 111, the flexible printed circuit layer 113 and the light-emitting units 12a, 12b can be prevented from touching directly the user U.

In this embodiment, the cap-body fixing member 134, provided to the inner surface A of the cap body 2, is configured to fix the second fixing member 132 at the first flexible substrate 111. The phototherapy lining 1 is then assembled to the cap body 2, such that the wearable phototherapy cap 100 according to this invention can be formed.

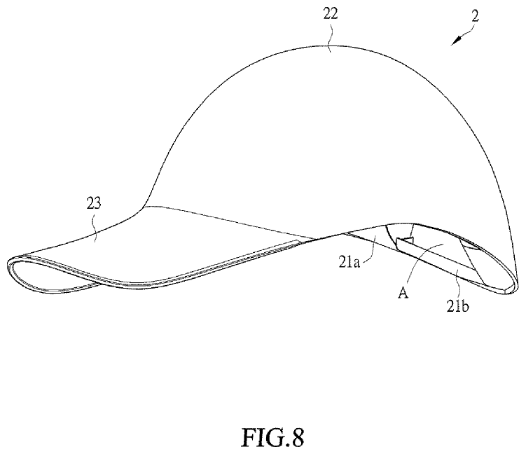

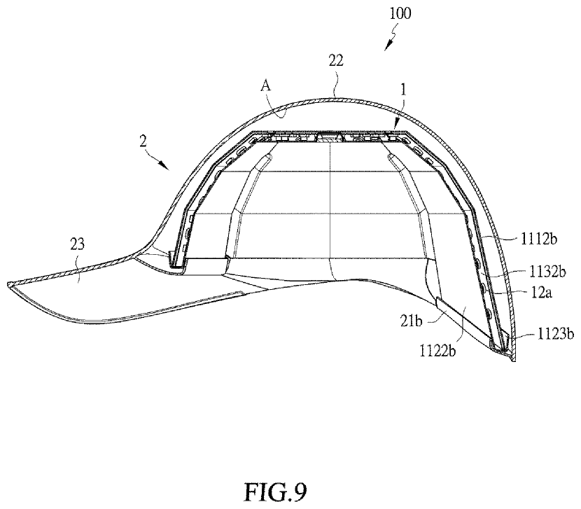

Refer to FIG. 1, FIG. 3 and FIG. 8 to FIG. 10 together; where FIG. 8 is a schematic perspective view of the cap body of the embodiment of FIG. 1, FIG. 9 is a schematic cross-sectional view of FIG. 1 along line B-B, and FIG. 10 demonstrates schematically a use state of the wearable phototherapy cap of FIG. 1. As shown, the cap body 2 further includes a plurality of wrap portions 21a, 21b (two labeled in the figure), preferably disposed on the inner surface A of the cap body 2.

The cap body 2 further includes a body portion 22. The wrap portions 21a, 21b would extend inward from the inner surface A of the body portion 22 so as to wrap auxiliarily at least part of the phototherapy lining 1. Further, the wrap portions 21a, 21b are provided in correspondence to the hook portions 1123a, 1123b so as to wrap auxiliarily part of the hook portions 1123a, 1123b and part of the second extension portions 1122a, 1122b. While the fixing assembly 13 fixes the phototherapy lining 1 and the cap body 2 at the composite central portion C, the wrap portions 21a, 21b would hold the hook portions 1123a, 1123b so as further to fix auxiliarily the phototherapy lining 1. In this embodiment, the cap body 2 further includes a visor portion 23 for shielding sun light and water drops.

As shown in FIG. 9, the flexible printed circuit layer 113 (labeled in FIG. 4) is disposed between the first flexible substrate 111 and the second flexible substrate 112, and the light-emitting units 12a, 12b are disposed on the flexible printed circuit layer 113 by positioning between the flexible printed circuit layer 113 and the second flexible substrate 112 so as to provide a plurality of beams to the second flexible substrate 112. The hook portion 1123b of the second flexible substrate 112 would be reflexed and extend further to wrap a portion of the first extension portion 1112b. Thereupon, the first extension portion 1112b and the third extension portion 1132b would be limited there by the hook portion 1123b, not to touch the user U. On the other hand, the wrap portion 21b of the cap body 2 would wrap auxiliarily the hook portion 1123b and part of the second extension portion 1122b. Thus, the second flexible substrate 112 would be held there so as to prevent the first extension portion 1112b, the third extension portion 1132b and the hook portion 1123b from further contacting directly the user U. In addition, the wrap portion 21b and the hook portion 1123b can be integrated to avoid the sweat of the user U to contaminate the flexible printed circuit layer 113, such that the flexible printed circuit layer 113 and the light-emitting units 12a, 12b can be better protected from further damages.

Thus, in the wearable phototherapy cap 100 provided by this invention, the structuring thereof is unique. Particularly, the composite substrate 11 of the phototherapy lining 1 is flexible and stretchable.

After the user U has the wearable phototherapy cap 100 on, the beams generated by the light-emitting units 12a, 12b would penetrate through the transparent second flexible substrate 112, and further onto the user U, such that the growth of hair of the user U would be substantially promoted. Practically, the flexible printed circuit layer 113 would be energized by a power supply so as to allow the light-emitting units 12a, 12b to generate the aforesaid light beams. Generally speaking, the power supply can be a general battery, a contact charging battery, a non-contact inductive charging battery, a portable power bank or any other type of power supplies. In addition, the power supply can be built-in or a foreign device.

As shown, the head of the user U can be divided into a first area HA1, a second area HA2 and a third area HA3. The first area HA1 is a top area, the second area HA2 is directed to a surrounding area above the ears, and the third area HA3 is a rear area lower than the ears. Namely, in a user U vision, the third area HA3 is a rear area adjacent to a neck of the user U. The light-emitting units 12a, 12b at the third central portion 1131 would irradiate the first area HA1 of the head. The light-emitting units 12a, 12b at the third extension portion 1132a and part of the light-emitting units 12a, 12b at the third extension portion 1132b would irradiate the second area HA2 of the head. The light-emitting units 12a, 12b at the lower third extension portion 1132b would irradiate the third area HA3 of the head. As such, the wearable phototherapy cap 100 can be applied to stimulate and thus promote the growth of the hair at the top of the head (the first area HA1), the hair surrounding the head (the second area HA2), and the hair at the lower rear of the head (the third area HA3).

In addition, the light beams provided by the light-emitting units 12a, 12b can be also applied to generate light beams for irradiating the user U for a purpose of head-skin treatment to promote healthy of the head skin, and further to promote the growth of the hair as well.

The wrap portions 21a, 21b are applied to wrap auxiliarily at least part of the phototherapy lining 1, such that the corresponding phototherapy lining 1 can be prevented from directly contacting the lower rear area of the head of the user U. In addition, the wrap portions 21a, 21b can also wrap auxiliarily the lower rear area, such that the wearable phototherapy cap 100 can cover a larger portion of the head of the user U.

In addition, according to this invention, the wearable phototherapy cap 100 is not simply applied to stimulate and promote the hair growth by providing the light-emitting units 12a, 12b to generate the light beams for irradiating the user U, but also to generate a quasi-massage function for releasing the muscle pressure at user's head and the area adjacent to user's neck. Namely, the wearable phototherapy cap 100 can relax the user U so as to promote the sleeping and to enhance the health of head skin. Since the wearable phototherapy cap 100 can provide benefits in relaxation and sleeping for the user U, thus conventional problems to modern people caused by abnormal work and rest can be substantially resolved. After the aforesaid problems from abnormal work and rest are solved, ill phenomena of insomnia, fatigue and headache can be lessened naturally. In addition, since the wearable phototherapy cap 100 is structurally resembled to a general baseball cap, thus the user U can wear the cap 100 comfortably.

In summary, in the wearable head phototherapy cap and the lining thereof provided by this invention, the first flexible substrate, the second flexible substrate, the flexible printed circuit layer, the light-emitting units and the fixing assembly are integrated for the user to stimulate and promote the hair growth. This invention further utilizes the hook portion of the second flexible substrate to reflex and further extend to wrap part of the first flexible substrate, such that the flexible printed circuit layer and the first flexible substrate can be free from contacting the user to cause discomfort. In addition, since the first flexible substrate, the second flexible substrate and the flexible printed circuit layer are flexible, and the first extension portion, the second extension portion and the third extension portion are separated to each other, thus the cap of the invention can fit various sizes of different users. Further, the wrap portion of the cap body is further used for wrapping auxiliarily part of the phototherapy lining, contacting the phototherapy lining, and thereby fixing auxiliarily the phototherapy lining and the cap body. The wrap portion and the hook portion are integrated to prevent user's sweat from entering the flexible printed circuit layer, and thus possible damages to the flexible printed circuit layer and the light-emitting units can be avoided. In addition, one of the third extension portions and the corresponding second extension portion are longer than the rest thereof, such that user's head (especially the lower rear portion) can be further irradiated. Further, the present invention can be worn outdoors, and thus the conventional shortcomings of laser equipment in medical institutions in higher cost, heavier weight, inability to carry out, limited use and inconvenience can be substantially resolved.

Furthermore, the present invention can also maintain the scalp (head skin), improve the scalp health, and promote the hair growth. In addition, the present invention can also relieve pressure on user's head, and thereby effects of relaxation and sleeping can be obtained.

While the present invention has been particularly shown and described with reference to a preferred embodiment, it will be understood by those skilled in the art that various changes in form and detail may be without departing from the spirit and scope of the present invention.

What is claimed is:

1. A phototherapy lining, assembled to an inner surface of a cap body, comprising:
a composite substrate, having a composite central portion, including:
a first flexible substrate, adhered to the inner surface;
a second flexible substrate, disposed away from the inner surface with respect to the first flexible substrate, being flexible and transparent, configured to be worn on a user's head, including:
a second central portion;
a plurality of second extension portions, extending from the second central portion; and
a plurality of hook portions, reflexed respectively from the plurality of second extension portions to wrap part of the first flexible substrate; and
a flexible printed circuit layer, disposed between the first flexible substrate and the second flexible substrate;
a plurality of light-emitting units, each of the plurality of light-emitting units being disposed between the flexible printed circuit layer and the second flexible substrate and configured to generate a light beam to irradiate the user's head; and
a fixing assembly, disposed at the composite central portion, configured for fixing the first flexible substrate, the flexible printed circuit layer and the second flexible substrate.

2. The phototherapy lining of claim 1, wherein the first flexible substrate further includes:
a first central portion; and
a plurality of first extension portions, extending from the first central portion.

3. The phototherapy lining of claim 2, wherein the flexible printed circuit layer further includes:
a third central portion; and
a plurality of third extension portions, extending from the third central portion.

4. The phototherapy lining of claim 3, wherein the plurality of first extension portions are separated to each other, the plurality of second extension portions are separated to each other, and the plurality of third extension portions are separated to each other.

5. The phototherapy lining of claim 4, wherein the second flexible substrate further includes a plurality of lateral extension portions, each of the plurality of lateral extension portions is extended toward the first flexible substrate from the corresponding second extension portion, and thereby the first flexible substrate and the second flexible substrate are integrated to wrap the flexible printed circuit layer.

6. The phototherapy lining of claim 3, wherein one of the plurality of third extension portions is longer than any of the rest of the plurality of third extension portions, and the second extension portion corresponding to the one of the plurality of third extension portions is longer than any of the rest of the plurality of second extension portions; wherein, while being applied to the user's head, one of the plurality of third extension portions is disposed in correspondence to a lower rear area of the user's head.

7. The phototherapy lining of claim 1, wherein the plurality of light-emitting units are disposed at the flexible printed circuit layer in an equal-spaced manner.

8. The phototherapy lining of claim 1, wherein the light beam has a wavelength ranging between 600 nm and 1000 nm.

9. A wearable phototherapy cap, to be worn by a user, comprising:
a cap body, having an inner surface; and
a phototherapy lining, assembled to the inner surface, including:
a composite substrate, having a composite central portion, including:
a first flexible substrate, adhered to the inner surface;
a second flexible substrate, disposed away from the inner surface with respect to the first flexible substrate, being flexible and transparent, configured to be worn on a user's head, including:
a second central portion;
a plurality of second extension portions, extending from the second central portion; and
a plurality of hook portions, reflexed respectively from the plurality of second extension portions to wrap part of the first flexible substrate; and
a flexible printed circuit layer, disposed between the first flexible substrate and the second flexible substrate;
a plurality of light-emitting units, each of the plurality of light-emitting units being disposed between the flexible printed circuit layer and the second flexible substrate and configured to generate a light beam to irradiate the user's head; and
a fixing assembly, disposed at the composite central portion, configured for fixing the first flexible substrate, the flexible printed circuit layer and the second flexible substrate to fix the phototherapy lining to the cap body.

10. The wearable phototherapy cap of claim 9, wherein the cap body further includes at least one wrap portion extending from a body portion of the cap body by opposing the inner surface, and the at least one wrap portion is configured to wrap auxiliarily at least part of the phototherapy lining, such that the at least part of the phototherapy lining is prevented from directly contacting a lower rear area of the user's head and used to cover auxiliarily the lower rear area.
